(19) United States
(12) Patent Application Publication
(10) Pub. No.: US 2024/0131196 A1
(43) Pub. Date: Apr. 25, 2024

(54) Systems and Methods to Enhance RNA Stability and Translation and Uses Thereof

(71) Applicant: The Board of Trustees of the Leland Stanford Junior University, Stanford, CA (US)

(72) Inventors: Rhiju Das, Palo Alto, CA (US); Hannah K. Wayment-Steele, Stanford, CA (US)

(73) Assignee: The Board of Trustees of the Leland Stanford Junior University, Stanford, CA (US)

(21) Appl. No.: 18/546,698

(22) PCT Filed: Jun. 30, 2021
(86) PCT No.: PCT/US21/40026

§ 371 Date: Aug. 15, 2023

Related U.S. Application Data
(60) Provisional application No. 63/149,939, filed on Feb. 16, 2021.

(51) Int. Cl. A61K 48/00

(52) U.S. Cl. CPC A61K 48/0066

(57) ABSTRACT

Embodiments herein describe systems and methods to enhance RNA stability and uses thereof. Many embodiments alter the sequence of an RNA therapeutic molecule (e.g., vaccines) to encode for a variant peptide while maintaining and/or increasing stability of the RNA therapeutic.

### CROSS-REFERENCE TO RELATED APPLICATIONS

The current application is a national stage of PCT Patent Application No. PCT/US2021/040026 entitled “Systems and Methods to Enhance RNA Stability and Translation and Uses Thereof” filed Jul. 1, 2021, which claims priority to U.S. Provisional Patent Application No. 63/149,939, filed Feb. 16, 2021; the disclosures of which are hereby incorporated by reference in their entirety.

### STATEMENT REGARDING FEDERALLY SPONSORED RESEARCH OR DEVELOPMENT

This invention was made with Governmental support under Contract Nos. GM122579 awarded by the National Institutes of Health. The government has certain rights in the invention.

### FIELD OF THE INVENTION

The present invention relates to ribonucleic acid (RNA). More specifically, the present invention relates to RNA molecules with enhanced stability and translation and further relates to systems and methods to enhance RNA stability and translation by selecting for structure of RNA molecules.

### BACKGROUND

There are multiple problems with prior methodologies of effecting protein expression. For example, introduced DNA can integrate into host cell genomic DNA at some frequency, resulting in alterations and/or damage to the host cell genomic DNA. Alternatively, the heterologous deoxyribonucleic acid (DNA) introduced into a cell can be inherited by daughter cells (whether or not the heterologous DNA has integrated into the chromosome) or by offspring.

In addition, assuming proper delivery and no damage or integration into the host genome, there are multiple steps which must occur before the encoded protein is made. Once inside the cell, DNA must be transported into the nucleus where it is transcribed into RNA. The RNA transcribed from DNA must then enter the cytoplasm where it is translated into protein. Not only do the multiple processing steps from administered DNA to protein create lag times before the generation of the functional protein, each step represents an opportunity for error and damage to the cell. Further, it is known to be difficult to obtain DNA expression in cells as DNA frequently enters a cell but is not expressed or not expressed at reasonable rates or concentrations. This can be a particular problem when DNA is introduced into primary cells or modified cell lines.

Attempts have been made to use RNA and messenger RNA (mRNA) as therapeutic agents. However, RNA is generally unstable and highly susceptible to degradation due to temperature, pH, and other factors.

### SUMMARY OF THE INVENTION

This summary is meant to provide some examples and is not intended to be limiting of the scope of the invention in any way. For example, any feature included in an example of this summary is not required by the claims, unless the claims explicitly recite the features. Various features and steps as described elsewhere in this disclosure may be included in the examples summarized here, and the features and steps described here and elsewhere can be combined in a variety of ways.

In one embodiment, a method for altering an RNA therapeutic sequence while maintaining stability includes obtaining a sequence of an RNA therapeutic, where the RNA therapeutic encodes for a first variant of a peptide, altering a codon sequence within the sequence of the RNA therapeutic, where the altered codon sequence changes the sequence of the RNA therapeutic to encode for a second variant of the peptide, and synthesizing an RNA molecule representing the altered sequence.

In a further embodiment, the altering step is performed by selecting a new codon for increased GC content relative to other codons for a particular amino acid, and substituting the new codon into the sequence for the RNA therapeutic to create a substituted coding sequence.

In another embodiment, the method further includes sampling a nucleotide within the target coding sequence within a certain distance to the codon sequence, where the sampled nucleotide includes an unpaired nucleotide within the coding sequence, and substituting the sampled nucleotide with a new nucleotide to create a substituted coding sequence.

In a still further embodiment, the method further includes treating an individual with the RNA molecule.

In still another embodiment, the method further includes formulating the RNA molecule for a medical use.

In a yet further embodiment, the formulating step includes combining the RNA molecule with one or more of a buffer, a lubricant, a binder, a flavorant, a coating, and an adjuvant.

In yet another embodiment, the method further includes encapsulating the RNA molecule.

In a further embodiment again, the capsule is selected from a virus, an adeno-associated virus, a viroid, a virion, a capsid, a micelle, a lipid nanoparticle, a DNA structure, and an RNA structure.

In another embodiment again, the method further includes substituting at least one nucleotide analog for a native nucleotide in the RNA molecule.

In a further additional embodiment, the nucleotide analog is selected from pseudouridine, inosine, 1-methyl-pseudouridine, and 5-methyl-cytidine, 1-methoxy-pseudouridine, and pseudo-isocytidine.

In another additional embodiment, the altered sequence possesses a lower DegScore than the RNA therapeutic, where DegScore=a*[stem nts]+b*[internal loop nts]+c*[hairpin nts]+d*[bulge nts]+e*[multiloop nts]+f*[exterior loop nts], where nts stands for nucleotides, and a-f represent coefficients for relative reactivity of nucleotides within a particular structure.

In a still yet further embodiment, an optimized RNA therapeutic includes an RNA molecule including a coding sequence having a codon change over a previous RNA therapeutic, where the codon encodes for a different amino acid than is encoded in the previous RNA therapeutic.

In still yet another embodiment, the codon has increased GC content relative to other codons to encode for the different amino acid.

In a still further embodiment again, the RNA molecule further includes a second codon change in a section of unpaired RNA sequence in the previous RNA therapeutic.

In still another embodiment again, the RNA molecule is formulated for medical use.

In a still further additional embodiment, the formulation further includes one or more of a buffer, a lubricant, a binder, a flavorant, a coating, and an adjuvant.

In still another additional embodiment, the RNA molecule is encapsulated.

In a yet further embodiment again, the capsule is selected from a virus, an adeno-associated virus, a viroid, a virion, a capsid, a micelle, a lipid nanoparticle, a DNA structure, and an RNA structure.

In yet another embodiment again, at least one nucleotide in the RNA molecule is substituted with a nucleotide analog.

In a yet further additional embodiment, the nucleotide analog is selected from pseudouridine, inosine, 1-methyl-pseudouridine, and 5-methyl-cytidine, 1-methoxy-pseudouridine, and pseudo-isocytidine.

In yet another additional embodiment, the RNA molecule possesses a lower DegScore than the previous RNA therapeutic, where DegScore=a*[stem nts]+b*[internal loop nts]+c*[hairpin nts]+d*[bulge nts]+e*[multiloop nts]+f*[exterior loop nts], where nts stands for nucleotides, and a-f represent coefficients for relative reactivity of nucleotides within a particular structure.

Other features and advantages of the present invention will become apparent from the following detailed description, taken in conjunction with the accompanying drawings which illustrate, by way of example, the principles of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The description and claims will be more fully understood with reference to the following figures and data graphs, which are presented as exemplary embodiments of the invention and should not be construed as a complete recitation of the scope of the invention.

FIG. 1A illustrates a method for altering an RNA therapeutic sequence in accordance with various embodiments of the invention.

FIG. 1B illustrates a schematic of altering codon sequences and stochastic sampling in accordance with various embodiments of the invention.

FIGS. 2A-2D illustrate various metrics and structures of small mRNAs in accordance with various embodiments of the invention.

FIGS. 3A-3C illustrate exemplary RNA structures designed rationally by participants during Eterna's OpenVaccine challenge result in the lowest AUP values for mRNAs encoding a variety of model proteins used for studying translation and as model vaccines, ranging in length from 144 nucleotides (the Multi-epitope Vaccine) to 855 nucleotides (eGFP+degron) in accordance with various embodiments of the invention. FIG. 3A illustrates force-directed graph visualization of sequences coding for eGFP+degron with lowest AUP value from each design source, colored by AUP per nucleotide. FIG. 3B illustrates that while ΔG(MFE) and AUP are correlated, the design with the lowest AUP is not the same as the design with the lowest ΔG(MFE). Starred points indicate the design for each design strategy with the lowest AUP value, calculated with ViennaRNA. FIG. 3C illustrates that Eterna designs show structural diversity as characterized by the Maximum Ladder Distance (MLD), the longest path of contiguous helices present in the MFE structure of the molecule. Additionally, FIG. 3C illustrates that MFE structures predicted in the ViennaRNA structure prediction package are depicted for designs a variety of MLD values, indicating similarly stabilized stems for a range of topologies.

FIGS. 4A-4C provide exemplary data illustrating that Eterna participants are able to design stabilized mRNAs for the SARS-CoV-2 full spike protein with the same degree of stabilization as in smaller mRNA design challenges in accordance with various embodiments of the invention. FIG. 4A shows participants were provided with metrics on a variety of structure metrics described in this work to aid in creating designs with a diversity of values. FIG. 4B shows solutions voted upon by the Eterna community show a diversity of structures while maintaining low AUP values. The solutions with the lowest AUP are structurally similar to and were derived from the dG(MFE) optimal structure from LinearDesign. FIG. 4C illustrates AUP values from different design methods are consistent across different mRNA lengths. A two-fold increase in lifetime is predicted by changing from a “Standard” design method (methods that do not stabilize structure) to a design method that increases structure.

FIG. 5 illustrates exemplary data of stabilization derived from low AUP solutions to small variations in protein sequence, variations in untranslated region (UTR), choice of folding algorithm, and predicted AUP at higher temperatures in accordance with various embodiments of the invention.

### DETAILED DESCRIPTION

Turning now to the drawings, systems and methods to enhance RNA stability and translation and uses thereof are provided. Many embodiments provide methods that provide an algorithmic approach to mutate an RNA sequence that optimizes stability and/or translation. In certain embodiments, the increased stability and/or translation is provided by increase in structure of the resultant RNA molecule.

There is a pressing need for vaccines against new viral pandemics like COVID-19, Ebola, flu, Zika, and other zoonotic viruses that jump from animal reservoirs into humans. mRNA molecules are considered one of the fastest ways to deploy these vaccines, but degrade and change their shape and effectiveness while stored in solution, even while refrigerated. Drug companies are not able to ship vaccines in pre-loaded syringes, making the logistical costs of deploying mass immunization currently prohibitive, and also incurring major safety risks.

A significant problem in RNA stability is self-cleavage, including from inline attack of 2′-hydroxyls on phosphates within an RNA. Stabilization of RNA molecules allows for mRNA and noncoding RNA molecules to remain active and/or intact across various environments, such as pre-filled syringes, such as could be used for RNA vaccines. In a variety of embodiments, the stable RNAs will be capable of space travel, environmental/agriculture applications, dissemination in animals or the human body, which could be used in biomedicine or human performance enhancement in extreme situations.

Messenger RNA (mRNA) molecules have shown promise as vaccine candidates in the current COVID-19 pandemic and may enable a large number of new therapeutic applications. (See e.g., Wu, F., et al. 2020. A New Coronavirus Associated With Human Respiratory Disease in China. Nature 579(7798):265-269; Chauhan, G., et al. Martinez-Chapa. Nanotechnology for COVID-19: Therapeutics and Vaccine Research. ACS Nano; McKay, P. F., et al. 2020. Self-amplifying RNA SARS-CoV-2 lipid nanoparticle vaccine candidate induces high neutralizing antibody titers in mice. Nature Communications 11(1):3523; Kaczmarek, J. C., P. S. Kowalski, and D. G. Anderson. 2017. Advances in the delivery of RNA therapeutics: from concept to clinical reality. Genome Medicine 9(1):60; Erasmus, J. H., and D. H. Fuller. 2020. Preparing for Pandemics: RNA Vaccines at the Forefront. Molecular Therapy 28(7):1559-1560; and Verbeke, R., I. Lentacker, S. C. De Smedt, and H. Dewitte. 2019. Three decades of messenger RNA vaccine development. Nano Today 28:100766; the disclosures of which are hereby incorporated by reference in their entireties.) However, a major limitation of mRNA technologies is the inherent chemical instability of RNA. mRNA manufacturing yields are reduced by degradation during in vitro transcription; mRNA vaccines stored in solution require in vitro stability, ideally over months under refrigeration; RNA vaccines deployed in developing regions would benefit from increased in vitro stability against high temperatures; and after being administered, mRNA vaccines require stabilization against hydrolysis and enzymatic degradation to sustain translation and immunogenicity in the human body. (See e.g., Zhao, P., et al. 2020. Long-term storage of lipid-like nanoparticles for mRNA delivery. Bioactive Materials 5(2):358-363; Organization, W. H. 2017. WHO preferred product characteristics for next generation influenza vaccines. World Health Organization, Geneva; and Zhang, N.-N., et al. 2020. A Thermostable mRNA Vaccine against COVID-19. Cell; the disclosures of which are hereby incorporated by reference in their entireties.)

RNA degradation depends on how prone the molecule is to in-line hydrolytic cleavage and attack by nucleases, oxidizers, and chemical modifiers in the RNA's environment. (See e.g., Markham, R., and J. D. Smith. 1952. The structure of ribonucleic acids. 1. Cyclic nucleotides produced by ribonuclease and by alkaline hydrolysis. Biochem J 52(4):552-557; Oivanen, M., S. Kuusela, and H. Lönnberg. 1998. Kinetics and Mechanisms for the Cleavage and Isomerization of the Phosphodiester Bonds of RNA by Brønsted Acids and Bases. Chemical Reviews 98(3):961-990; Cataldo, F. 2006. Ozone Degradation of Biological Macromolecules: Proteins, Hemoglobin, RNA, and DNA. Ozone: Science & Engineering 28(5):317-328. Journal Article; and Baldridge, K. C., J. Zavala, J. Surratt, K. G. Sexton, and L. M. Contreras. 2015. Cellular RNA is chemically modified by exposure to air pollution mixtures. Inhalation Toxicology 27(1):74-82. Journal Article; the disclosures of which are hereby incorporated by reference in their entireties.) Amongst these degradation processes, in-line hydrolytic cleavage is a universal mechanism intrinsic to RNA. Cleavage of an RNA backbone phosphodiester bond is initiated by deprotonation of the 2′-hydroxyl group of the ribose moiety. (See e.g., Li, Y., and R. R. Breaker. 1999. Kinetics of RNA Degradation by Specific Base Catalysis of Transesterification Involving the 2′-Hydroxyl Group. J. Amer. Chem. Soc. 121(23):5364-5372; the disclosure of which is hereby incorporated by reference in its entirety.)

Hydrolysis sets a fundamental limit on the stability of mRNA medicines and technologies. The World Health Organization's target product profile for vaccines calls for formulations that remain effective after a month under refrigeration. Deployment of mRNA vaccines for infectious disease outbreaks, like the COVID-19 pandemic, would benefit from taking advantage of existing supply networks for conventional attenuated vector vaccines, which are set up for pre-filled syringes in saline buffer at near-neutral pH under refrigeration. Model calculations of RNA hydrolysis as a function of pH, temperature, and ionic concentration, highlight potential problems for using the same supply networks for mRNA vaccines. (See e.g., Kaukinen, U., S. Lyytikäinen, S. Mikkola, and H. Lönnberg. 2002. The Reactivity of Phosphodiester Bonds Within Linear Single-Stranded Oligoribonucleotides Is Strongly Dependent on the Base Sequence. Nucleic Acids Res. 30(2); the disclosure of which is hereby incorporated by reference in its entirety.) Under refrigerated transport conditions (‘cold-chain’, 5° C., phosphate-buffered saline, pH 7.4, no Mg2+), a naked RNA molecule encoding a SARS-CoV-2 spike glycoprotein, with a length of roughly 4000 nucleotides in bulk solution would have a half-life of 900 days, with 98% intact after 30 days, fitting the target product profile for vaccines from the World Health Organization. However, a temperature excursion to 37° C. is predicted to lead to a half-life reduced to 5 days, well under a month. Even if temperature can be maintained at 5° C., RNAs encapsulated in lipid formulations may be subject to increased hydrolysis if the lipid's cationic headgroups lower the pKa of the ribose 2-hydroxyl group (17, 18). If pKa shifts as small as 2 units occur, the predicted half-life reduces from 900 days to 10 days, again well under a month (Table 1). Beyond the above considerations for a ˜4000 nt mRNA, the longer lengths of RNA molecules being considered for low-material-cost ‘self-amplifying’ mRNA (SAM) vaccines are expected to exacerbate inline hydrolysis. (See e.g., Geall, A. J., et al. 2012. Nonviral delivery of self-amplifying RNA vaccines. Proc. Natl. Acad. Sci. 109(36);); the disclosure of which is hereby incorporated by reference in its entirety.) In all conditions described above, the half-life will be reduced by a further 3-fold compared to a non-SAM mRNA. As an example, if during storage or shipment at pH 7.4, the SAM vaccine of length 12,000 nts is subject to an excursion of temperature to 37° C. for 2 days, the fraction of functional, full-length mRNA remaining after that excursion will drop to less than half of the starting RNA (Table 1). Beyond these calculations under storage and shipping conditions, an mRNA vaccine is expected to be highly unstable during in vitro transcription and upon delivery into the human body (half-lives reduced to hours due to presence of Mg²⁺ and physiological temperatures; Table 1).

Many embodiments employ a largely unexplored design method to reduce RNA hydrolysis that is largely independent of mRNA manufacturing, formulation, storage, and in vivo conditions. Such embodiments increase the degree of secondary structure present in the RNA molecule. As described herein, hydrolysis is mitigated by the presence of secondary structure, which restricts the possible conformations the backbone can take and reduces the propensity to form conformations prone to in-line attack. (See e.g., Mikkola, S., U. Kaukinen, and H. Lönnberg. 2001. The Effect of Secondary Structure on Cleavage of the Phosphodiester Bonds of RNA. Cell Biochem. Biophys. 34(1); the disclosure of which is hereby incorporated by reference in its entirety.) Indeed, the technique of inline probing takes advantage of the suppression of in-line hydrolysis within double-stranded or otherwise structured regions to map RNA structure. (See e.g., Regulski, E. E., and R. R. Breaker. 2008. In-line Probing Analysis of Riboswitches. Methods Mol. Biol. 419; the disclosure of which is hereby incorporated by reference in its entirety.)

Many embodiments describe a framework and computational results indicating that structure-aware design enables immediate and significant COVID-19 mRNA vaccine stabilization. Various embodiments provide a principled model that links an RNA molecule's overall hydrolysis rate to base-pairing probabilities, which are readily calculated in widely used modeling packages. (See e.g., Lorenz, R., et al. 2011. ViennaRNA Package 2.0. Algorithms Mol Biol 6:26; Zadeh, J. N., et al. 2011. NUPACK: Analysis and design of nucleic acid systems. J Comput Chem 32(1):170-173; Reuter, J. S., and D. H. Mathews. 2010. RNAstructure: software for RNA secondary structure prediction and analysis. BMC Bioinformatics 11:129; and Do, C. B., D. A. Woods, and S. Batzoglou. 2006. CONTRAfold: RNA secondary structure prediction without physics-based models. Bioinformatics 22(14):e90-98; the disclosures of which are hereby incorporated by reference in their entireties.) Various embodiments identify specific metrics: the Summed Unpaired Probability of a molecule, or SUP, and the Average Unpaired Probability, or AUP, which is the SUP normalized by sequence length. By comparing a variety of mRNA design methods, certain embodiments show that rational design and stochastic optimization algorithms are able to minimize AUP for the CDS across a range of mRNA applications. Such embodiments predict that structure-optimizing designs can achieve at least two-fold increases in estimated mRNA half-life, independent of the mRNA length. Additionally, many embodiments optimize mRNA half-life while retaining other desirable sequence or structure properties of the mRNA, such as codon optimality, short stem lengths, and compactness measures, which may modulate in vivo mRNA translation and immune response.

A biophysical model for RNA degradation. Previous studies have explored the design of mRNA molecules with increased secondary structure, as evaluated by the predicted folding free energy of the mRNA's most stable structure, but it is unclear if this metric is the correct one when improving stability of an RNA against degradation. (See e.g., Terai, G., S. Kamegai, and K. Asai. 2016. CDSfold: An Algorithm for Designing a Protein-Coding Sequence With the Most Stable Secondary Structure. Bioinformatics 32(6); Zhang, H., et al. 2020. LinearDesign: Efficient Algorithms for Optimized mRNA Sequence Design. Arxiv; and Cohen, B., and S. Skiena. 2003. Natural Selection and Algorithmic Design of mRNA. Journal of Computational Biology 10(3-4); the disclosures of which are hereby incorporated by reference in their entireties.) However, many embodiments are based on a principled model of RNA degradation that suggests an alternative metric. To describe the degradation rate of an RNA molecule, such embodiments imagine that each nucleotide at position i has a rate of degradation k_{cleavage}(i). it is imagined that the degradation is due to inline hydrolysis, but the framework below generalizes to any degradation process that is suppressed by formation of RNA structure, including endonuclease digestion. The probability that the nucleotide backbone remains intact at nucleotide i after time t is:

p_{intact}(i,t)=e^{-k}^{_{cleavage}}^{(i)t}. (1)

The probability that the overall RNA with length N remains intact with no chain breaks after time t is then:

p_{intact} ^{overall}(t)=Πᵢ₌₁^{N}p_{intact}(i,t)=Πᵢ₌₁^{N}e^{-k}^{_{cleavage}}^{(i)t}=exp (−Σᵢ₌₁^{N}k_{cleavage}(i)t). (2)

Here, it is assumed that the probability of cleavage at any given position is independent of cleavage events at other positions in the RNA. If this is not true, the expression will still remain correct at times when there are 0 or 1 cleavage events, and that is the time range most relevant for improving RNA stability. Given that assumption, eq. (2) gives an exactly exponential dependence of the overall degradation of the RNA with respect to time t:

p_{intact}^{overall}(t)=e^{-k}^{_{cleavage}}^{overall}^{ₜ}, (3)

with:

k_{cleavage}^{overall}=Σᵢ₌₁^{N}k_{cleavage}(i). (4)

The degradation half-life of the RNA is t_{1/2}=ln 2/k_{cleavage}^{overall}.

The rate at which an RNA is hydrolyzed at a specific location along its backbone k_{cleavage}(i) depends on the ability of the phosphodiester bond to adopt the in-line attack conformation, or more generally for the RNA to adopt a conformation that can be accessed by a degrading agent, like a protein nuclease. Here and below, the consequences of a simple model reflecting the knowledge that in-line hydrolysis generally occurs at nucleotides that are unpaired but is strongly suppressed by pairing of the nucleotide into double-stranded segments of the secondary structure. Since RNA chains fluctuate between multiple secondary structures with a characteristic timescale of milliseconds, faster than the degradation rates (estimated in Table 1), the overall cleavage rate as averaged over the equilibrated structural ensemble of the RNA can be written:

k_{cleavage}^{overall}=Σ_{s∈{S}}p(s)Σᵢk_{cleavage}(i|s), (5)

where {S} is the full set of structures that the RNA molecule is capable of adopting, and p(s) is the probability of forming a structure s. The rate of cleavage k_{cleavage}(i|s) at each position i within a structure s will, in general have a complex dependence on the sequence and structural context. For example, the cleavage rate will depend on whether the nucleotide is in a hairpin loop, where it is in within the loop, whether other loop nucleotides might promote in-line cleavage through acid-base catalysis, whether the loop has non-canonical pairs, etc. Without additional empirical knowledge, it is assumed that the cleavage rate for unpaired nucleotides can be approximated by a constant rate k_{cleavage}^{unpaired} if nucleotide i is unpaired, and zero if paired. Then eq. (5) becomes:

$$\begin{matrix}{k}_{cleavage}^{overall} & = {\sum }_{s ∈ \{ S \}} {\sum }_{i} p ( s ) {k}_{cleavage} ( i \vert s ) \\ & = {\sum }_{i} {\sum }_{s ∈ \{ S \}} p ( s ) {k}_{cleavage} ( i \vert s ) \\ & = {\sum }_{i} {\sum }_{s ∈ \{ S \}} p ( s ) I ( i unpaired in s ) {k}_{cleavage}^{unpaired} \\ & = {k}_{cleavage}^{unpaired} {\sum }_{i} {p}_{unpaired} ( i ) \\ & = {k}_{cleavage}^{unpaired} \times SUP ,\end{matrix} \qquad ( 6 )$$

where I(i unpaired in s) is 1 if nucleotide i is unpaired in the structure s and 0 otherwise. In the last line of (6), the definition of Sum of Unpaired Probabilities is introduced:

SUP=Σᵢp_{unpaired}(i). (7)

Overall, the total rate of cleavage may be approximated as this measure, the sum of unpaired probabilities across all nucleotides of the RNA, multiplied by a constant k_{cleavage}^{unpaired} that reflects the average cleavage rate of an unpaired nucleotide.

The total rate scales with the sum of the unpaired probabilities of the RNA's nucleotides—longer RNA molecules are expected to degrade faster in proportion to their length. This relation is better reflected by a rearrangement of (6) to:

kₒᵥₑᵣₐₗₗ^{unpaired}=k_{cleavage}^{unpaired}×N×AUP (8)

where the average unpaired probability (AUP) is:

$$AUP = \frac{1}{N} {\sum }_{i} {p}_{unpaired} ( i ) = \frac{1}{N} SUP . \qquad ( 9 )$$

The AUP value is a number between 0 and 1 that reflects the overall ‘structuredness’ of the RNA. Lower values correspond to lower probability of being unpaired, and therefore RNA molecules less susceptible to degradation.

In these last expressions (eqns. 6-9), p_{unpaired}(i) can be predicted in most widely-used RNA secondary structure prediction packages, which output base pair probabilities p(i:j), the probability that bases i and j are paired. Then the probability that any nucleotide i is unpaired in the RNA is given as:

SUP=Σᵢ₌₁^{N}[1−Σⱼ₌₁^{N}p(i:j)], AUP=SUP/N. (10)

Under this model, it becomes possible to computationally study the question of how much an RNA might be stabilized if it is redesigned to form stable secondary structures.

Additionally, based on nucleotide reactivity, a degradation score can be determined and/or predicted for a particular sequence based on the predicted structure for an RNA sequence. The following equation provides on formula for calculating a degradation score (DegScore), in accordance with some embodiments:

DegScore=a*[stem nts]+b*[internal loop nts]+c*[hairpin nts]+d*[bulge nts]+e*[multiloop nts]+f*[exterior loop nts], (11)

Where nts stands for nucleotides, and a-f represent coefficients for relative reactivity of nucleotides within a particular structure. In many embodiments, the coefficients range from 0.0-1.0 (e.g., if nucleotides in exterior loops are 5× more reactive than nucleotides in an internal loop, coefficient b could equal 0.2, while coefficient f could equal 1.0).

### Maintaining RNA Stability while Changing Coding Sequence

Many embodiments are directed to systems and methods to change the coding sequence of an RNA therapeutic. For example, such embodiments are important at times when an initial vaccine has reduced efficacy to a mutated or novel strain. Such embodiments can alter the coding sequence of an RNA therapeutic while maintaining the designed stability of the therapeutic.

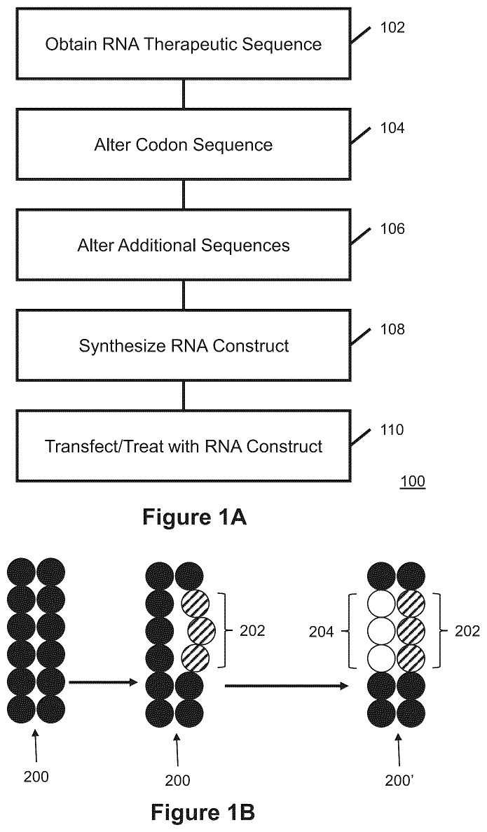

Turning to FIG. 1A, a method 100 to alter the coding sequence of an RNA is illustrated. In various embodiments, function is defined as increased stability and/or translation. Increased RNA stability includes reduced degradation of the RNA in vivo, in vitro, in storage, during manufacture, or any combination thereof. At 102, many embodiments obtain an RNA therapeutic sequence. In various embodiments, the RNA sequence comprises only a coding sequence, while in some embodiments, the RNA sequence comprises a coding sequence coupled with functional segments, including a poly-A tail, a 5′ untranslated region (5′UTR), a 3′ untranslated region (3′UTR), and/or any other sequence to assist in RNA function. Various embodiments obtain an RNA sequences that has been designed for a therapeutic. In various embodiments, the therapeutic sequence has been optimized for increased stability, including through increased base pairing, secondary structure, modified nucleotides, nucleotide analogs and/or any other methodology for increasing RNA stability.

At 104, many embodiments alter one or more codon sequences in the RNA, so the RNA codes for a variant. For example, a codon may be altered to change an amino acid in the resultant peptide or protein. In some embodiments, all possible codons to code for the variant are obtained. In certain embodiments, multiple codons are altered, to alter more than one amino acid in the resultant peptide. In some embodiments, the codon is selected based on the GC content of the codon. Certain embodiments select a codon with increased GC content over other possible codons for a particular amino acid—for example, UCC or UCG may be selected to code for serine, rather than UCU or UCA. Various embodiments retain the codon variant with the lowest DegScore (see Eqn. 11).

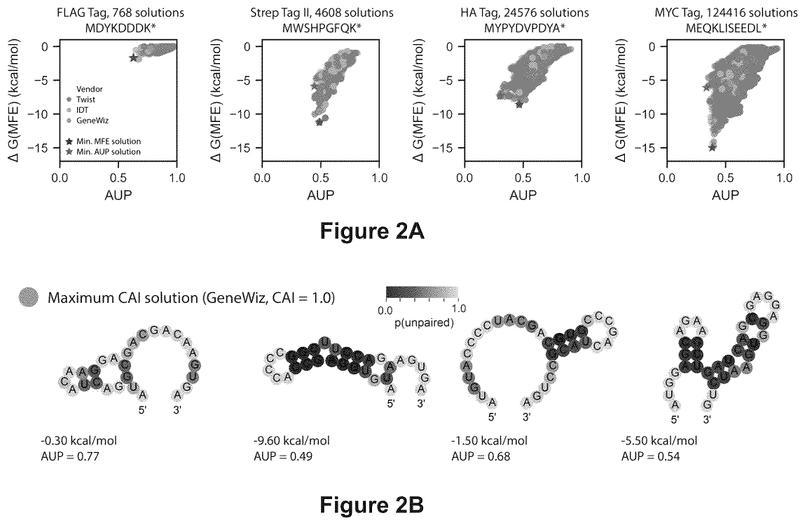

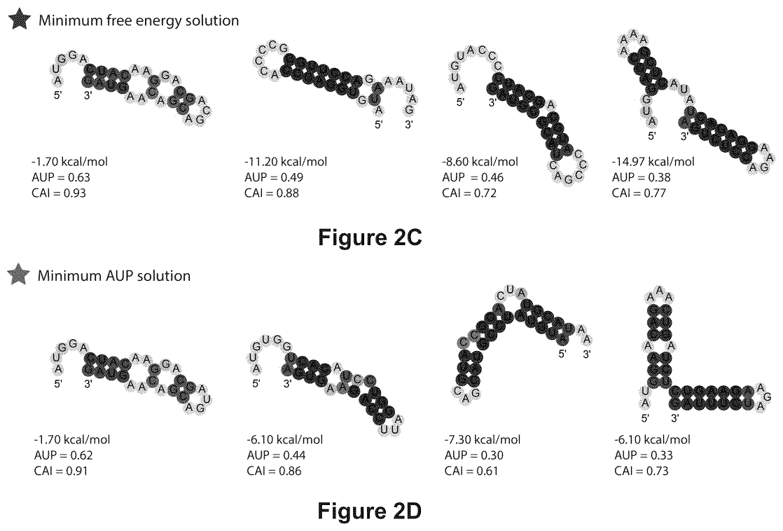

At 106, the RNA sequence is altered at additional locations of various embodiments. Certain embodiments perform stochastic sampling at additional sites within the RNA sequence. In some embodiments, the stochastic sampling involves altering sequences that pair with the altered codon in a way to maintain the coding of the paired sequences while maintaining or increasing stability of the RNA molecule. FIG. 1B illustrates examples of such stochastic sampling, where RNA sequence 200 is altered at codon 202, which causes codon 200 to not pair in a stem structure. Stochastic sampling at sequence 204 may allow the return of pairing, thus possibly increasing stability of the new sequence 200′. While FIG. 2 illustrates an example of stochastic sampling in the context of a stem structure, such sampling can occur in other structures or conformations, including loops, junctions, and any other structure within the RNA. Certain embodiments perform synonymous substitutions for certain codons. For example, a codon may be altered to increase the GC content without changing the resultant amino acid.

Returning to FIG. 1A, further embodiments synthesize an RNA construct representing the altered RNA sequence at 108. Various embodiments synthesize the RNA construct chemically via various known technologies, while additional embodiments synthesize the RNA nanostructure via biochemical. Example methods of synthesis include phosphoramidite chemistry, T7 polymerase, and any other known or applicable means of synthesizing an RNA construct. In various embodiments, the oligonucleotides include just the developed path from a starting point to an ending point, while in some embodiments, the oligonucleotide includes a portion (including the entirety) of the molecule at the starting point and/or a portion (including the entirety) of the molecule at the ending point. Certain embodiments synthesize the construct using RNA base pairs, while some embodiments synthesize the construct using DNA base pairs, and additional embodiments synthesize the construct using a combination of RNA and DNA base pairs. Further, embodiments synthesize the oligonucleotide double stranded, single stranded, or a combination of double and single stranded. Certain embodiments incorporate nucleotide analogs into sequences to increase mRNA stability, including pseudouridine, inosine, 1-methyl-pseudouridine, and 5-methyl-cytidine, 1-methoxy-pseudouridine, and pseudo-isocytidine, and other known analogs.

At 110 of many embodiments, an RNA construct is transfected into a cell and/or someone is treated. As noted elsewhere herein, RNA constructs can have many purposes, including coding for reporter genes, vaccines, other RNAs for translation, and functional RNAs (e.g., small RNAs, interfering RNAs, ribosomal RNAs, and any other functional RNAs). As such, transfecting a cell of certain embodiments inserts the RNA directly, such as through microinjection, particle bombardment, electroporation or other direct means. In certain embodiments involving the treatment of an individual, an RNA construct can be formulated for a medical use, including by combining it with one or more buffers, lubricants, binders, flavorants, adjuvants, and coatings. Various embodiments encapsulate the RNA construct for specific transfection, such as through a virus (e.g., adeno-associated viruses (AAVs)), viroids, virions, capsids, micelles, lipid nanoparticles, and/or larger DNA and/or RNA structures suitable for targeting and/or stability.

### EXEMPLARY EMBODIMENTS

Although the following embodiments provide details on certain embodiments of the inventions, it should be understood that these are only exemplary in nature, and are not intended to limit the scope of the invention.

### Example 1: Small mRNA Models Reveal Discrepancy in Sequences Optimized for SUP vs. Sequences Optimized for Codon Optimality or Minimum Folding Free Energy

Background: To investigate the possible dynamic range in degradation lifetimes for mRNA, this embodiment started with mRNA design problems that were small enough to be tractable, i.e., all mRNA sequences that code for the target amino acid sequence could be directly enumerated and studied.

Methods: A collection of short peptide tags were selected that are commonly appended or prepended to proteins to enable purification or imaging: His tags of varying lengths, human influenza hemagglutinin (HA) tag, Strep-tag II, FLAG fusion tag, and Myc tag sequences. All the mRNA sequences that encode each protein were enumerated. For reference, sequences were also generated from online codon optimization tools from gene vendors IDT, GENEWIZ, and Twist.

Results: As expected, the predicted degradation rates of solutions from these vendor sequences, which do not consider mRNA in vitro stability, were higher than the minimal degradation rate computed for all enumerated sequences (FIG. 2A). The change in AUP obtained between the average of all the vendor-returned sequences and the minimum AUP solution for tag proteins was calculated. Fold-changes in AUP ranging from 1.27 to 2.23-fold decrease in AUP were also identified (Table 2).

Interestingly, it was discovered that minimal-AUP solutions were close to optimal with respect to other metrics previously considered in mRNA design. The codon adaptation index (CAI), or related metrics that match codons frequency of the designed mRNA to mRNA's in the target organisms, are optimized by vendor design algorithms; the GENEWIZ algorithm gives a CAI of 1.0. The minimum AUP solutions have CAI values of 0.91, 0.86, 0.61, and 0.73 respectively, which are comparable to vendor-generated sequences, suggesting that optimization of AUP will not result in a major penalty on CAI.

In other studies, structured mRNAs have been designed through optimization of the predicted folding free energy of the minimum free energy structure (MFE), which can be achieved through an exact algorithm. For 4 of the 10 model systems studied—the three His tags and the FLAG tag—the coding sequence with the lowest free energy MFE structure also exhibited the lowest AUP (FIG. 2A). For other model proteins, including the Strep-tag II, the HA tag, and the Myc tag, the coding sequence with the lowest-energy MFE structure was not the same as the solution with the lowest AUP (FIG. 2A). The most notable difference was in designs for the HA tag: the lowest AUP value obtained was 0.30, which is 1.5-fold lower than AUP of the minimal MFE solution, 0.46. However, inspection of the two solutions clarifies why a structure with a higher free energy but a lower AUP would be preferred if we wish to reduce overall hydrolysis (FIGS. 2C-2D). Minimal AUP solutions have more stems and fewer ‘hot spots’ (5 vs. 15 yellow nucleotides in HA panel of FIGS. 2D vs. 2C) rather than optimize the folding free energy of each stem, once formed (reflected here in the base pairing probability; magenta vs. dark purple coloring in HA panel of FIGS. 2D vs. 2C). Repeating the same analysis across other small model systems as well as with other secondary-structure packages reveals similar results.

Conclusion: Taken together, this enumerative analysis of small model mRNAs suggested up to 2-fold increased stabilization might be achievable in mRNA design while retaining excellent codon adaptation indices and that solutions with minimal folding free energy are not necessarily expected to be most stable to hydrolysis.

### Example 2: A Two-Fold Decrease in AUP is Achievable for Long mRNA Constructs

Background: To test the applicability of the insights from small peptide-encoding mRNAs (Example 1) to more realistic protein-encoding mRNA design problems, mRNAs with lengths of hundreds of nucleotides encoding a variety of target proteins were tested, some with therapeutic potential against SARS-CoV-2 and some commonly used in laboratory settings and animal studies to test protein synthesis levels. The four systems were a multi-epitope vaccine design (MEV) derived from SARS-CoV-2 spike glycoprotein (S) and nucleocapsid (N) proteins; Nanoluciferase; enhanced green fluorescence protein with an attached degron sequence (eGFP+deg) for characterizing mRNA stability and translation; and the SARS-CoV-2 spike receptor binding domain (RBD) of the SARS-CoV-2 spike protein.

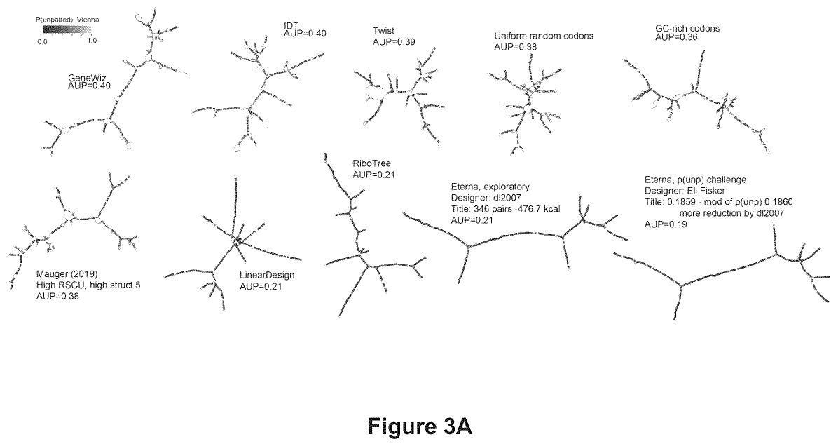

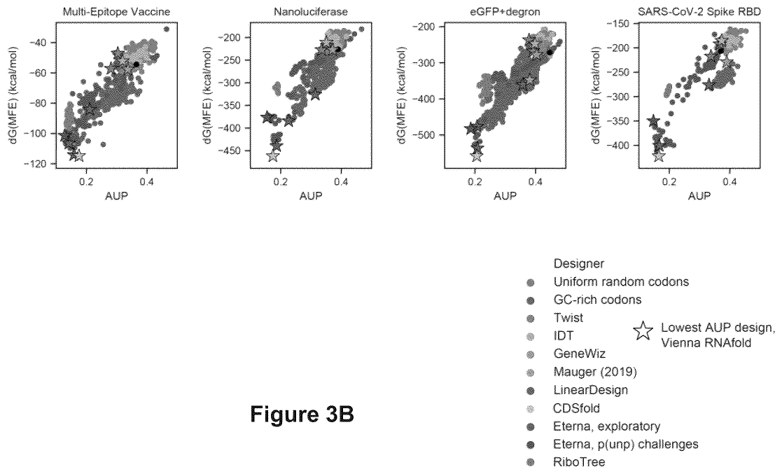

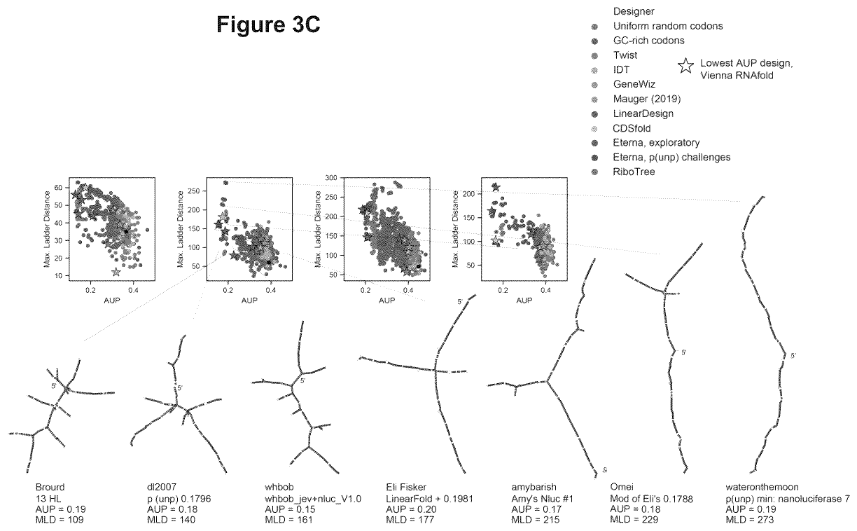

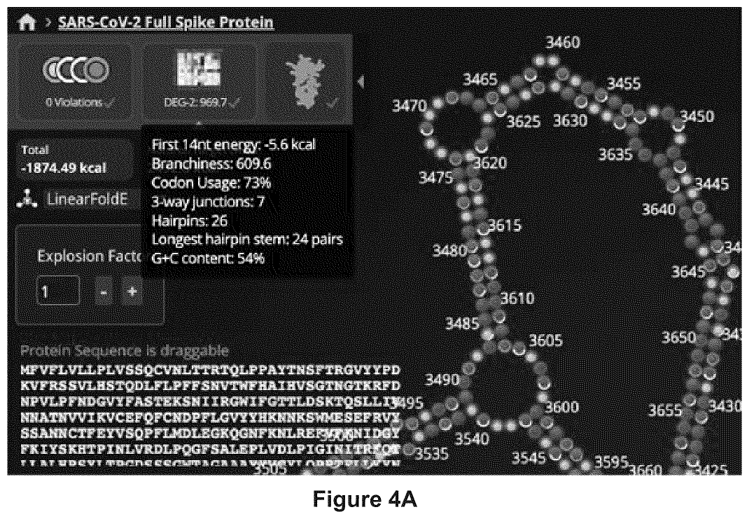

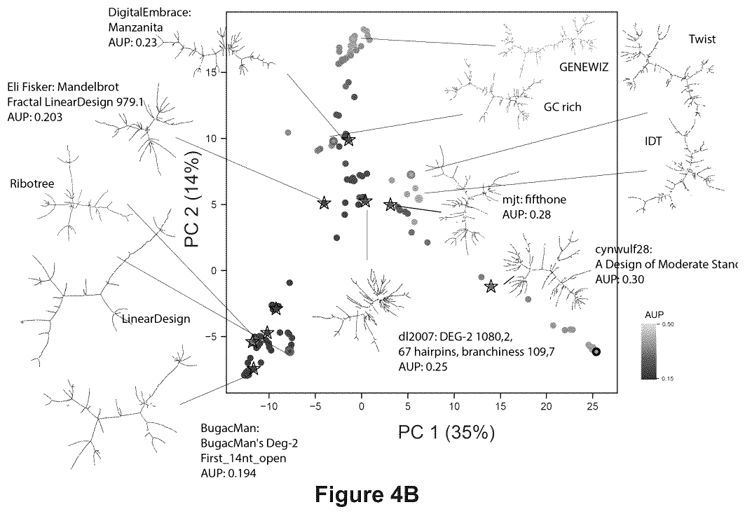

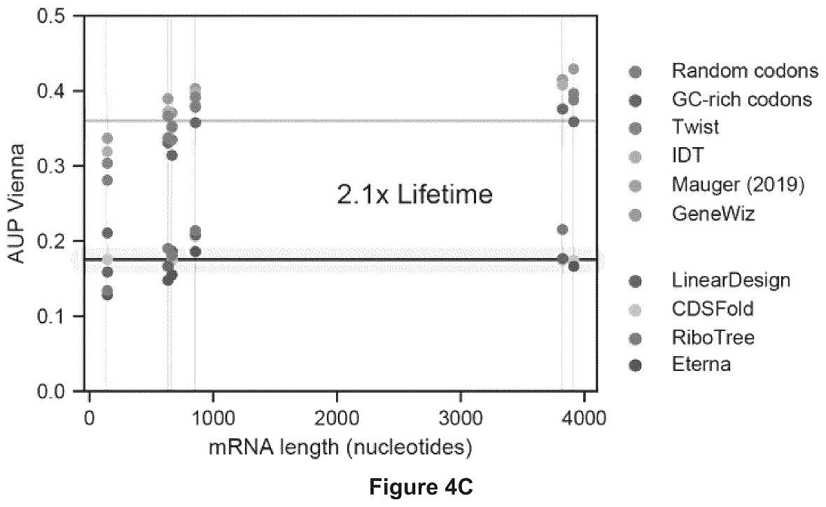

Methods: Because enumeration of mRNA sequences is not possible for these problems, sequences generated by a variety of methods described herein were compared: uniform sampling of codons (“Uniform random codons”); uniform sampling of GC-rich codons only (“GC-rich codons”); vendor-supplied servers from IDT, GENEWIZ, and Twist; the algorithm CDSfold, which returns a sequence with minimal ΔG(MFE) solution; the algorithm LinearDesign, which also returns a minimal ΔG(MFE) solution that is balanced with codon usage, as well as sequences from other groups when possible. A stochastic Monte Carlo Tree Search algorithm to stochastically minimize AUP of model mRNAs was further developed. Last, crowdsourced solutions through the online RNA design platform Eterna were also used. Early Eterna challenges, labeled “Eterna, exploratory” in FIGS. 3 and 4, were not set up with any specific optimization targets other than a general call to create mRNAs that coded for the target proteins but formed significant structures in Eterna's game interface, which provides folding calculations in a number of secondary structure prediction packages. An additional set of Eterna sequences were solicited in the “p(unp) challenges”, where the AUP metric was calculated and provided to Eterna participants within the game interface to guide optimization.

Results: It was found that designs from the tested algorithmic and crowdsourcing approaches encompassed a wide range of sequence space, and that sequences with low AUP values did not localize to specific regions of sequence space.

For all four challenges, the sequences with the lowest AUP values were designed by Eterna participants. FIG. 3A depicts MFE structures of the minimal AUP sequence for each design method for the eGFP+degron challenge (the longest mRNA), with nucleotides colored by their unpaired probability, as calculated in the ViennaRNA folding package. Structures portrayed in FIG. 3A indicate visual hallmarks of structures with lower AUP: solutions from LinearDesign, RiboTree, and Eterna have longer helices, fewer loops and junctions, and lower unpaired probabilities in stems (indicated by dark purple). Notably, the solutions with the minimal AUP were distinct from solutions with the lowest ΔG(MFE) (FIG. 3B) for all four challenges. Table 3 contains summary statistics for AUP values for design methods separated by standard methods (codon sampling, gene vendor tools) and methods intended to stabilize secondary structure (Eterna p(unp) rational design, CDSfold, LinearDesign, etc.).

The values of AUP achieved by Eterna participant submissions in the “p(unp) challenge” (mean and standard deviations of MEV: 0.22±0.08, Nluc: 0.24±0.08, eGFP: 0.28±0.08, Spike RBD: 0.24±0.08) were significantly lower than values from standard methods, including codon random sampling and vendor-generated sequences (MEV: 0.34±0.04, Nluc: 0.36±0.02, eGFP: 0.40±0.02, Spike RBD: 0.39±0.02, Table 3). The lowest AUP values from Eterna participants (MEV: 0.128, Nluc: 0.155, eGFP: 0.186, Spike RBD: 0.148) were lower in each case than the AUP values of LinearDesign constructs, (MEV: 0.159, Nluc: 0.186, eGFP: 0.208, Spike RBD: 0.167), CDSfold constructs (MEV: 0.160, Nluc: 0.160, eGFP: 0.206, Spike RBD: 0.165), or of minimum AUP solutions from the novel stochastic software (MEV: 0.134, Nluc: 0.181, eGFP: 0.214, Spike RBD: 0.190). The novel stochastic software came closest (within 5%) to the minimal Eterna AUP value for the shortest mRNA sequence, suggesting that RiboTree was better able to search sequence space for the shorter sequences. One of the challenges, the eGFP+degron mRNA, could be compared to designs developed by others based on folding free energy optimization to increase functional mRNA lifetime in human cells. The minimal AUP value from those sequences (0.381) was similar to the value obtained from randomly-sampled codons, indicating that explicit optimization of AUP rather than folding free energy is necessary for applications seeking stability against hydrolysis. Repeating these analyses of mRNAs with other secondary-structure packages reveals similar results.

Conclusion: Solutions generated by the novel stochastic software exhibited low AUP while not necessarily minimizing ΔG(MFE). Minimum AUP solutions from RiboTree had absolute ΔG(MFE) values that were up to 75% reduced (less stable) compared to absolute ΔG(MFE) values of minimum ΔG(MFE) solutions, which came from Eterna participants (MEV: 93%, Nanoluciferase: 82%, eGFP+deg: 75%, Spike RBD: 84%). Minimizing AUP without minimizing ΔG(MFE) may prove to be a valuable design strategy for developing mRNAs that are stable under storage but need to be sufficiently unstable as to exhibit cooperative unfolding by the cells' translational apparatus.

### Example 3: Diversity of Properties Related to Translation and Immunogenic Function

Background: After establishing the feasibility of designing mRNA sequences with reduced AUP, it was desired to determine if these sequences might be viable for translation and for either preventing or eliciting innate immune responses.

Methods and Results: In advance of experimental tests, sequence and structure properties that have been hypothesized to correlate with translation and immunogenicity were tabulated. Firstly, the sequences designed in the above challenges (See Example 2) did not contain untranslated regions (UTRs), and it was ascertained that the overall AUP of a designed coding sequence (CDS) also maintains low AUP in the context of UTRs. It was found that for the collected sequence designs, the AUP calculated in the context of standard human betaglobin (HBB) UTRs had near-perfect correlation to the AUP of the CDS only.

Next, the codon adaptation index (CAI) of sequences across design methods was characterized, as this measure has been implicated in improving translation efficiency. We found that across all p(unp) challenges, minimal AUP sequences consistently had CAI values greater than 0.7. Another design feature that has been hypothesized to influence protein translation efficiency is the exposure of the CDS immediately upstream of the initiation codon. The summed unpaired probabilities of the first 14 nucleotides were calculated, termed SUP^{init,14}, in the presence of our model UTRs (HBB). A higher value of SUP^{init,14} indicates a more exposed ribosome initiation site, and is implicated to correlate with higher translation efficiency. A range of SUP^{init} values possible for low AUP sequences were found. These analyses suggest that it is feasible to design low AUP sequences that are translatable, at least as assessed by the widely-used metric of CAI and SUP^{init,14}.

Another important consideration is the possibility of mRNA therapeutics eliciting immunogenic responses from pathways that recognize double-stranded RNA helices. It was found that none of the sequences characterized included helices longer than 33 base pairs, a measure that has been found to be the minimum length that leads to global shutdown of cellular mRNA translation after sensing by protein kinase R. Importantly, however, different low-AUP designs exhibited a wide spectrum of helix lengths (8 to 22 base pairs) suggesting that a less drastic innate immune response might be achieved, and be tunable depending on whether such responses are desirable (mRNA vaccines) or not (e.g., for anti-immune mRNA therapeutics).

In addition to structural characteristics that affect stability against in vitro hydrolysis, translatability and degradation rates in cells, and immunogenicity of mRNA molecules, it is expected that there are many structural characteristics that relate to a molecule's in vivo persistence that are not yet well understood. The ability to design multiple low-AUP sequences with a large range of alternative structures increases the potential that a functional design may be found in empirical tests or as the connections between mRNA structure and function are better understood. For instance, in FIG. 3A, it was observed that although LinearDesign, RiboTree, and Eterna sequences for an eGFP+degron mRNA all have AUP values within 10% of each other, they have different secondary structures. The same can be seen for all the mRNA design problems tested.

As a more quantitative evaluation of structural diversity, the Maximum Ladder Distance (MLD) of designed sequences were characterized. This measure has been used to describe the compactness of viral genomic RNAs and has been hypothesized to be relevant for viral packaging, immunogenicity, and biological persistence. If an RNA molecule's secondary structure is represented as an undirected graph, where edges represent helices, edge lengths correspond to helix lengths, and vertices correspond to loops, the MLD is the longest path that can be traced in the graph. Genomic viral RNAs have been demonstrated to have shorter MLDs than equivalent random controls, and molecules with shorter MLDs have been shown to be more compact experimentally, a feature that may also contribute to persistence. It was found that AUP and MLD were negatively correlated across the MEV, Nanoluciferase, eGFP+degron, and Spike RBD challenges (−0.64, −0.59, −0.62, −0.70, respectively, FIG. 3C). This overall (negative) correlation reflects how minimizing AUP leads to larger average MLD values. Nevertheless, it is noted that the MLD values still fall over a wide range for sequences with low AUP. Example structures from the Nanoluciferase challenge are depicted at the bottom of FIG. 3C, the challenge that showed the widest range of MLD values for low AUP structures, which range from highly branched, compact structures (FIG. 3C, bottom-left) to long, snake-like structures (FIG. 3C, bottom-right). These structures exhibit uniformly low unpaired probabilities in stems (indicated by dark purple coloring), with the main difference being the layout of stems. In addition to MLD, we calculated several other metrics characterizing structure, such as counts of different types of loops and junctions, the ratio of number of hairpins to number of 3-way junctions in the MFE structure, as a measure of branching, and mean distance between nucleotides in base pairs. In all cases, values ranged by over 2-fold in low-AUP solutions, underscoring the diversity of structures that can be achieved.

Conclusion: These results demonstrate that both automated and rational design methods are capable of finding RNA sequences with low AUP values but a wide range of diverse structures. Testing these mRNAs experimentally for their translation rates and persistence in cells and in animals will help address the relationship between MLD and mRNA therapeutic stability.

### Example 4: Eterna Participants are able to Design Stabilized SARS-CoV-2 Full Spike Protein mRNAs

Background: For longer mRNA design problems, including the SARS-CoV-2 spike protein mRNA used in COVID-19 vaccine formulations (3822 nts), it is noted that the computational cost associated with computing thermodynamic ensembles associated with AUP became slow and hindered automated or interactive design guided by AUP. Therefore, other observables were sought that were more rapid to compute to guide design of RNA's stabilized against hydrolysis.

Methods and Results: Correlations between many observables and AUP were calculated, and found that for all four challenges, the number of unpaired nucleotides in the single MFE structure was the most correlated with AUP, giving near-perfect correlations (0.98, 0.99, 0.99, 0.99, respectively). This observation was leveraged to launch another design puzzle on Eterna in July 2020: minimizing the number of unpaired nucleotides in the MFE structure, as a proxy for AUP, for a vaccine design that includes the full SARS-CoV-2 spike protein (“JEV+Spike”). It was found that Eterna participants were capable of finding values for AUP as low as in previous challenges, despite the fact that the JEV+Spike mRNA was over four times as long as previous challenges. Again, this solution was distinct from the minimal dGMFE design calculated in CDSFold. The lowest AUP value for the JEV+Spike protein was 0.166, 2.5-fold lower than the minimum AUP values from conventional design methods (0.40 +/−0.02). The novel stochastic method was also run minimizing the number of unpaired nucleotides. The larger size of the JEV+Spike protein meant that it took longer for the novel stochastic method to minimize the solution. Starting from a random initialization and running the novel stochastic method for 6000 iterations (2 days) resulted in a construct with an AUP of 0.254. Seeding the novel stochastic method with a starting sequence partially stabilized using the LinearDesign (CITE) server (AUP: 0.212) resulted in reducing the AUP (0.206).

When the SARS-CoV-2 spike protein sequence used in vaccine formulations was publicly disclosed, one more puzzle was launched on Eterna calling for solutions for stabilized mRNAs encoding the S-2P protein. By this point, other predictors for RNA degradation trained on high-throughput data had been developed. Participants were not specifically asked to minimize any of the several metrics provided, (AUP, the number of unpaired nucleotides, or DegScore). Participants were provided with a variety of metrics described in this work (FIG. 4A). Out of 181 submissions, the top 9 solutions that were voted upon demonstrated a diverse set of sequences, some prioritizing structure diversity, some prioritizing high stability, all demonstrating low AUP values (FIG. 4B).

Conclusion: As with shorter mRNAs, S-2P solutions with the lowest AUP values—from Eterna participants, the novel stochastic method, CDSfold, and LinearDesign—demonstrate a 2-fold reduction in AUP from mRNAs designed through randomly selecting codons or from codon optimization algorithms from gene synthesis vendors (FIG. 4C).

### Example 5: Highly Stable mRNAs are Robust to Variations in Design

Background: There are several design contexts in which it would be advantageous to adapt a designed highly-stable mRNA rather than design a new mRNA de novo.

Methods and Results: The robustness of designed S-2P mRNA stability to small changes in protein sequence was tested, reflecting the potential need for booster vaccines for variant strains and to different UTRs, reflecting different expression platforms and formulations. The 9 top-voted sequences from the Eterna S-2P round were selected, as well as one representative sequence from other methods (Twist, IDT, GENEWIZ, GC-rich, LinearDesign, CDSfold, etc.) to test these sequence mutations and UTR variations.

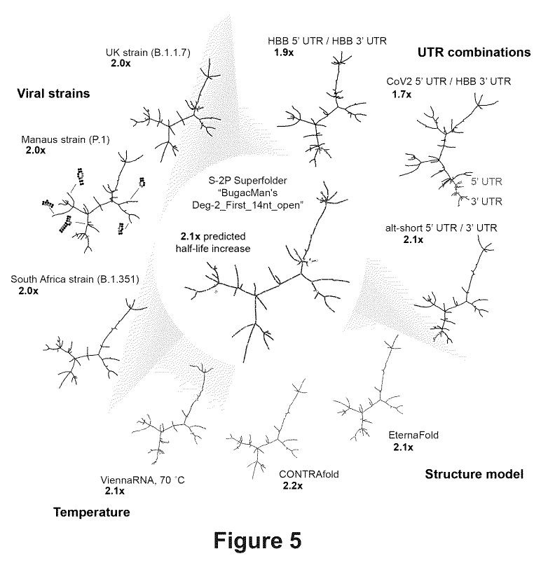

A heuristic was developed to adapt a m RNA design for a mutation of the original coded protein. For each amino acid mutation, the new codon was replaced with the most GC-rich codons for the mutant. This heuristic was used to design mRNA sequences coding for the UK strain (B.1.1.7), the Manaus strain (P.1), and the South Africa strain (B.1.351). It was found that for the selection of both stabilized and conventionally-designed mRNAs tested, the AUP of the modified mRNA had near-perfect correlation with the AUP of the original mRNA (0.999, 0.996, 0.999, for B.1.1.7, P.1, B.1.351, respectively). The addition of mutations did not perturb the global layout of the mRNA design (example for one sequence depicted in FIG. 5).

To test the effect of adding different UTRs, AUP was calculated for the subset of sequences in the context of three UTR combinations studied in the context of the OpenVaccine consortium: 1. human hemoglobin beta (HBB) 5′/3′ UTR; 2) a modification of the SARS-CoV-2 5′ UTR (CoV-2-TTG-TTGfull-¬¬-1-dSL1-3) and the Dengue Virus 3′ UTR; and 3) the 5′ UTR from complement factor 3 and the 3′ UTR from the Sindbis Virus URE element. These were selected to represent a diversity of UTR structuredness and length. The AUP of the full constructs also had very high correlation to the AUP of the CDS only (0.999, 0.993, 0.992, respectively).

Conclusion: Stabilization derived from low AUP solutions is robust to small variations in protein sequence, variations in untranslated region (UTR), choice of folding algorithm, and predicted AUP at higher temperatures.

### DOCTRINE OF EQUIVALENTS

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the foregoing examples and descriptions of various preferred embodiments of the present invention are merely illustrative of the invention as a whole, and that variations in the components or steps of the present invention may be made within the spirit and scope of the invention. Accordingly, the present invention is not limited to the specific embodiments described herein, but, rather, is defined by the scope of the appended claims.

TABLE 1
Estimates for RNA degradation AUP

| | | | | | | Cleavage rate | |
|---|---|---|---|---|---|---|---|
| Simulated condition | | | [Mg²⁺] | RNA length | | per molecule | Half-life^{b} |
| (0.14M [K⁺]) | T (° C.) | pH | (mM) | (nucleotides) | AUP^{a} | (k_{deg}) (10⁻⁷ min⁻¹) | (days) |
| Refrigerated supply | 5 | 7.4 | 0 | 4,000 | 0.4 | 5.1 | 941 |
| chain (‘cold chain’) | | | | | | | |
| Refrigerated supply | 5 | 7.4 | 0 | 12,000 | 0.4 | 15.3 | 314 |
| chain, increased | | | | | | | |
| length (SAM RNA) | | | | | | | |
| Refrigerated supply | 5 | 9.4^{c} | 0 | 4,000 | 0.4 | 470 | 10.2 |
| chain, pKa shifted by | | | | | | | |
| cationic formulation | | | | | | | |
| Temperature | 37 | 7.4 | 0 | 4,000 | 0.4 | 890 | 5.4 |
| excursion | | | | | | | |
| Manufacturing (in | 37 | 7.6 | 14 | 4,000 | 0.4 | 57,000 | 0.084 |
| vitro transcription) | | | | | | | |
| Physiological | 37 | 7.4 | 1 | 4,000 | 0.4 | 2,000 | 2.4 |

^{a}Standard AUP of 0.4 estimated from standard design methods studied in this work.
^{b}Calculated as t_{1/2} = ln 2/k_{deg}.
^{c}Apparent pH at 2′ hydroxyl, assuming pKₐ shift of 2 units induced by complexation with cationic lipid.

TABLE 2
AUP values for example tag proteins and calculated fold-change decrease
in AUP from standard design methods to the global minimum AUP value.

| | FLAG Tag | Strep Tag II | HA Tag | MYC Tag |
|---|---|---|---|---|
| Protein length | 8 | 9 | 10 | 11 |
| mRNA CDS length^{a} | 27 | 30 | 33 | 36 |
| # Synonymous mRNAs | 768 | 4,608 | 24,576 | 124,416 |
| Standard methods^{b}, mean(std). AUP | 0.86(7) | 0.57(7) | 0.67(9) | 0.60(6) |
| Global Min. AUP | 0.62 | 0.44 | 0.30 | 0.33 |
| Mean standard/Global min. | 1.39 | 1.30 | 2.23 | 1.82 |

^{a}mRNA length = 3 × protein length + 3 (stop codon).
^{b}GENEWIZ, IDT, Twist.

TABLE 3
Statistics of AUP values obtained in comparing different
classes of design methods on mRNA design challenges.

| | Multi- | | | | | |
|---|---|---|---|---|---|---|
| | epitope | Nano- | eGFP + | | | |
| mRNA design challenge | vaccine | luciferase | degron | Spike RBD | JEV + Spike | S-2P |
| Protein length (aa) | 47 | 221 | 284 | 210 | 1303 | 1273 |
| mRNA CDS length (nt) | 144 | 666 | 855 | 633 | 3912 | 3822 |
| Standard methods^{b}, | 0.34(4) | 0.36(2) | 0.40(2) | 0.39(2) | 0.40(2) | 0.40(2) |
| mean(std) AUP | | | | | | |
| Stabilizing methods^{c}, | 0.15(2) | 0.17(2) | 0.20(1) | 0.17(2) | 0.18(2) | 0.19(2) |
| mean(std) AUP | | | | | | |
| Stabilizing methods^{c}, min. | 0.13 | 0.15 | 0.19 | 0.15 | 0.17 | 0.17 |
| AUP^{d} | | | | | | |
| Mean standard/min | 2.6 | 2.4 | 2.1 | 2.6 | 2.4 | 2.4 |
| stabilized | | | | | | |

^{a}mRNA length = 3 × protein length + 3 (stop codon).
^{b}Uniform codons, GC codons, GENEWIZ, IDT, Twist.
^{c}Eterna p(unp) challenge, RiboTree, LinearDesign.
^{d}In all cases, minimal AUP was achieved in Eterna p(unp) challenge.

What is claimed is:

1. A method for altering an RNA therapeutic sequence while maintaining stability comprising:
obtaining a sequence of an RNA therapeutic, wherein the RNA therapeutic encodes for a first variant of a peptide;
altering a codon sequence within the sequence of the RNA therapeutic, wherein the altered codon sequence changes the sequence of the RNA therapeutic to encode for a second variant of the peptide; and
synthesizing an RNA molecule representing the altered sequence.

2. The method of claim 1, wherein the altering step is performed by:
selecting a new codon for increased GC content relative to other codons for a particular amino acid; and
substituting the new codon into the sequence for the RNA therapeutic to create a substituted coding sequence.

3. The method of claim 1, further comprising:
sampling a nucleotide within the target coding sequence within a certain distance to the codon sequence, wherein the sampled nucleotide comprises an unpaired nucleotide within the coding sequence; and
substituting the sampled nucleotide with a new nucleotide to create a substituted coding sequence.

4. The method of claim 1, further comprising treating an individual with the RNA molecule.

5. The method of claim 4, further comprising formulating the RNA molecule for a medical use.

6. The method of claim 5, wherein the formulating step comprises combining the RNA molecule with one or more of: a buffer, a lubricant, a binder, a flavorant, a coating, and an adjuvant.

7. The method of claim 4, further comprising encapsulating the RNA molecule.

8. The method of claim 7, wherein the capsule is selected from: a virus, an adeno-associated virus, a viroid, a virion, a capsid, a micelle, a lipid nanoparticle, a DNA structure, and an RNA structure.

9. The method of claim 1, further comprising substituting at least one nucleotide analog for a native nucleotide in the RNA molecule.

10. The method of claim 9, wherein the nucleotide analog is selected from pseudouridine, inosine, 1-methyl-pseudouridine, and 5-methyl-cytidine, 1-methoxy-pseudouridine, and pseudo-isocytidine.

11. The method of claim 9, wherein the altered sequence possesses a lower DegScore than the RNA therapeutic, wherein
DegScore=a*[stem nts]+b*[internal loop nts]+c*[hairpin nts]+d*[bulge nts]+e*[multiloop nts]+f*[exterior loop nts],
where nts stands for nucleotides, and
a-f represent coefficients for relative reactivity of nucleotides within a particular structure.

12. An optimized RNA therapeutic comprising:
an RNA molecule comprising a coding sequence having a codon change over a previous RNA therapeutic, wherein the codon encodes for a different amino acid than is encoded in the previous RNA therapeutic.

13. The optimized RNA therapeutic of claim 12, wherein the codon has increased GC content relative to other codons to encode for the different amino acid.

14. The optimized RNA therapeutic of claim 12, wherein the RNA molecule further comprises a second codon change in a section of unpaired RNA sequence in the previous RNA therapeutic.

15. The optimized RNA therapeutic of claim 12, wherein the RNA molecule is formulated for medical use.

16. The optimized RNA therapeutic of claim 15, wherein the formulation further comprises one or more of: a buffer, a lubricant, a binder, a flavorant, a coating, and an adjuvant.

17. The optimized RNA therapeutic of claim 12, wherein the RNA molecule is encapsulated.

18. The optimized RNA therapeutic of claim 17, wherein the capsule is selected from: a virus, an adeno-associated virus, a viroid, a virion, a capsid, a micelle, a lipid nanoparticle, a DNA structure, and an RNA structure.

19. The optimized RNA therapeutic of claim 12, wherein at least one nucleotide in the RNA molecule is substituted with a nucleotide analog.

20. The optimized RNA therapeutic of claim 19, wherein the nucleotide analog is selected from: pseudouridine, inosine, 1-methyl-pseudouridine, and 5-methyl-cytidine, 1-methoxy-pseudouridine, and pseudo-isocytidine.

21. The optimized RNA therapeutic of claim 12, wherein the RNA molecule possesses a lower DegScore than the previous RNA therapeutic, wherein
DegScore=a*[stem nts]+b*[internal loop nts]+c*[hairpin nts]+d*[bulge nts]+e*[multiloop nts]+f*[exterior loop nts],
where nts stands for nucleotides, and
a-f represent coefficients for relative reactivity of nucleotides within a particular structure.
